(19) United States
(12) Patent Application Publication
(10) Pub. No.: US 2011/0294726 A1
(43) Pub. Date: Dec. 1, 2011

(54) ANTIBIOTIC THERAPY TO REDUCE THE LIKELIHOOD OF DEVELOPING POST-INFECTIOUS IRRITABLE BOWEL SYNDROME

(75) Inventors: Mark Pimentel, Los Angeles, CA (US); Christopher Chang, Los Angeles, CA (US)

(73) Assignee: CEDARS-SINAI MEDICAL CENTER, Los Angeles, CA (US)

(21) Appl. No.: 13/148,215

(22) PCT Filed: Feb. 11, 2010
(86) PCT No.: PCT/US10/23873

§ 371 Date: Aug. 5, 2011

Related U.S. Application Data
(60) Provisional application No. 61/151,772, filed on Feb. 11, 2009.

(51) Int. Cl. A61K 38/12; A61P 31/04; A61K 31/4164; A61K 31/437; A61K 31/7036

(52) U.S. Cl. 514/2.9; 514/279; 514/39; 514/398

(57) ABSTRACT

The present invention provides for methods of preventing IBS, preventing long term irregular bowel pattern, reducing the likelihood of developing or having IBS, reducing the likelihood of developing or having long term irregular bowel pattern, mitigating IBS, mitigating long term irregular bowel pattern and reducing the likelihood of developing non-ulcer dyspepsia. The methods comprise providing an antibiotic and administering the antibiotic to a subject in need thereof.

### FIELD OF INVENTION

This invention relates to methods of reducing the likelihood of developing irritable bowel syndrome.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Irritable bowel syndrome (IBS) is a chronic gastrointestinal condition characterized by altered bowel function and abdominal pain. Although the pathophysiology of IBS remains unknown, multiple theories have emerged. One theory is that a form of IBS is initiated by acute gastroenteritis (1-8). This is termed post-infectious IBS. In a recent meta-analysis of studies prospectively examining the incidence of IBS after outbreaks of acute gastroenteritis, the development of IBS was seen to occur in 9.8% of subjects (9). Although initially there was some speculation that the follow-up of subjects in studies of post-infectious IBS were too brief to determine chronicity, Neale et al. demonstrated that 6 years after the onset of IBS, 57% of subjects deemed post-infectious IBS continued to meet Rome criteria for IBS (10). In this condition of post-infectious IBS, one demonstrable hallmark of the condition is an increase in rectal lymphocytes (11).

In a recent animal study, the inventors' group showed that acute inoculation of rats with Campylobacter jejuni (“C. jejuni”) resulted in a phenotype of altered stool form, 3 months after the clearance of C. jejuni from the stool of the animals (12). In addition, the rats demonstrated small intestinal bacterial overgrowth as defined by quantitative PCR. Rats with small intestinal bacterial overgrowth were more likely to have altered stool form. Interestingly, this model mimicked the human condition further by demonstrating a significant increase in rectal lymphocytosis. This model is important since it is derived from a pathogen commonly known to occur in human acute gastroenteritis. It also allows a more detailed study of the pathogenesis of post-infectious IBS.

In the human condition, antibiotic use for acute gastroenteritis is a risk factor for the development of IBS (9). However, this could possibly be related to referral bias. The sicker a patient is, the more likely they are to be given antibiotics. This is supported by data showing that the more severe the case of gastroenteritis, the more likely IBS will develop (9). Recently, efforts have been made to examine antibiotic prophylaxis in humans traveling to endemic areas for organisms such as Escherichia coli (13).

While many people can control their IBS symptoms (e.g., cramping, abdominal pain, bloating, constipation, and diarrhea), with diet, stress management, and medications, for some people IBS can be disabling. They may be unable to work, attend social events, or even travel short distances. As many as 20% of the adult population have symptoms of IBS, making it one of the most common disorders diagnosed by doctors. Accordingly, there exists a need for a treatment to prevent and/or reduce the likelihood of having or developing IBS.

Described herein, the inventors examined the effect of prophylactic antibiotic therapy during acute C. jejuni inoculation of animals. Specifically, the inventors determined whether this prophylactic treatment will prevent the development of chronic altered stool form long after clearance of C. jejuni in a rat model of post-infectious IBS.

### SUMMARY OF THE INVENTION

The following embodiments and aspects thereof are described and illustrated in conjunction with compositions and methods which are meant to be exemplary and illustrative, not limiting in scope.

The present invention provides a method, comprising: identifying a subject selected from the group consisting of: a subject who desires a reduction of the likelihood of developing or having post infectious irritable bowel syndrome (PI-IBS), a subject who desires a reduction of the likelihood of developing or having long term irregular bowel pattern, a subject who desires a mitigation of PI-IBS that may develop, a subject who desires a mitigation of long term irregular bowel pattern, a subject who desires a reduction of the likelihood of developing or having non-ulcer dyspepsia (NUD), a subject in need of reducing the likelihood of developing or having post infectious irritable bowel syndrome (PI-IBS), a subject in need of reducing the likelihood of developing or having long term irregular bowel pattern, a subject in need of mitigating PI-IBS that may develop, a subject in need of mitigating long term irregular bowel pattern, a subject in need of reducing the likelihood of developing or having NUD, and combinations thereof; providing an antibiotic; and administering the antibiotic to the subject to reduce the likelihood of developing or having PI-IBS, to reduce the likelihood of developing or having long term irregular bowel pattern, to mitigate PI-IBS that may develop, to mitigate long term irregular bowel pattern and/or to reduce the likelihood of developing or having NUD in the subject.

In various embodiments, the antibiotic may be a non-absorbable antibiotic, such as, rifaximin.

In one embodiment, the subject does not have small intestinal bacterial overgrowth (SIBO). In various embodiments, the subject has not taken an antibiotic to treat an intestinal infection, to prevent an intestinal infection, to reduce the likelihood of having an intestinal infection, to treat a gastric infection, to prevent a gastric infection or to reduce the likelihood of having a gastric infection.

In another embodiment, the subject may be exposed to a higher risk of having food poisoning or gastroenteritis. In various embodiments, the food poisoning or gastroenteritis may be caused by Campylobacter, such as, Campylobacter jejuni. In other embodiments, the food poisoning or gastroenteritis may be caused by Escherichia coli, Salmonella or Shigella.

The present invention also provides for a method, comprising: identifying a subject in need of inhibiting the production of cytolethal distending toxin (CDT) and/or inhibiting the interaction of CDT with an intestinal cell; providing an antibiotic; and administering the antibiotic to the subject to inhibit the production of CDT and/or inhibit the interaction of CDT with the intestinal cell. In various embodiments, inhibiting the production of CDT and/or inhibiting the interaction of CDT with the intestinal cell reduces the likelihood of developing or having post infectious irritable bowel syndrome (PI-IBS), reduces the likelihood of developing or having long term irregular bowel pattern, mitigates PI-IBS that may develop, mitigates long term irregular bowel pattern, and/or reduces the likelihood of developing or having non-ulcer dyspepsia (NUD).

In various embodiments, the antibiotic may be a non-absorbable antibiotic, such as, rifaximin.

In one embodiment, the subject does not have small intestinal bacterial overgrowth (SIBO). In other embodiments, the subject has not taken an antibiotic to treat an intestinal infection, to reduce the likelihood of having an intestinal infection, to treat a gastric infection, and/or to reduce the likelihood of having a gastric infection. In another embodiment, the subject may be exposed to a higher risk of having food poisoning or gastroenteritis. In one embodiment, the food poisoning or gastroenteritis may be caused by Campylobacter, such as, Campylobacter jejuni. In other embodiments, the food poisoning or gastroenteritis may be caused by Escherichia coli, Salmonella or Shigella.

The present invention also provides for a method, comprising: identifying a subject who is being treated with a first antibiotic or will be treated with the first antibiotic; and administering a second antibiotic selected from the group consisting of rifaximin, neomycin, metronidazole, vancomycin and combinations thereof to reduce the subject's likelihood of having a Clostridium difficile infection. In various embodiments, reducing the subject's likelihood of having a Clostridium difficile infection reduces the subject's likelihood of developing or having irritable bowel syndrome (IBS), reduces the subject's likelihood of developing or having long term irregular bowel pattern, mitigates IBS that may develop in the subject, mitigates long term irregular bowel pattern for the subject, and/or reduces the likelihood of developing or having non-ulcer dyspepsia (NUD).

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts C. jejuni colonization of stool in rats with (C+/R+) and without prophylactic rifaximin (C+/R−) in accordance with an embodiment of the present invention.

FIG. 2 depicts the number of bowel movements that were normal in form out of 3 days in rats that had not received (C+/R−) and those that received (C+/R+) rifaximin in accordance with an embodiment of the present invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Mammal” as used herein refers to any member of the class Mammalia, including, without limitation, humans and nonhuman primates such as chimpanzees, and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus adult and newborn subjects, as well as fetuses, whether male or female, are intended to be included within the scope of this term.

“Irregular bowel pattern” as used herein refers to a change in the consistency of stool form and/or a change in the frequency of bowel movements.

In the human condition of post-infectious IBS, studies have demonstrated that almost 10% of subjects that were otherwise normal develop IBS following acute gastroenteritis (9). While the initial descriptions of post-infectious IBS were in subjects who were infected with C. jejuni, the development of IBS has since been shown to occur with a number of bacterial pathogens including E. coli, Salmonella and Shigella (1-9). These studies support a potential causative factor in IBS for the first time.

In addition to the altered stool form and meeting of Rome criteria for IBS, subjects with post-infectious IBS also demonstrate one notable mucosal characteristic. Specifically, there appears to be a slight but significant elevation in rectal lymphocytes (11). In a study by Spiller et al., the number of intraepithelial lymphocytes per 100 epithelial cells among subjects developing post-infectious IBS is 1.8 compared to 0.3 in healthy control subjects. However, the study of post-infectious IBS in humans is limited by access to tissue, and an animal model is needed to better elucidate the physiologic and molecular features of post-infectious IBS.

To date, the most widely published model of post-infectious gastrointestinal sequelae in an animal model utilized the pathogen Trichinella spiralis. In this model, numerous important findings have been made. Although a recent outbreak in Turkey demonstrated that Trichinella spiralis in humans can lead to post-infectious IBS (Soyturk et al., Irritable bowel syndrome in persons who acquired trichinellosis. AM J GASTROENTEROL. 2007; 102:1064-9.), this pathogen is uncommon in humans and thus, is not a common cause of post-infectious IBS.

In a recently published paper, the inventors' group described a post-infectious model in rats (Pimentel et al., A new rat model links two contemporary theories in irritable bowel syndrome. DIG DIS SCI 2008; 53:982-9). In this study, 3 months after rats were gavaged and C. jejuni cleared from their stool, they demonstrated altered stool form. In addition, 27% of the rats had small intestinal bacterial overgrowth and this overgrowth predicted the rats more likely to have altered stool form. The rats also demonstrated an increase in the intraepithelial lymphocyte count in the rectum and left colon but not cecum or small bowel. In this new rat model using C. jejuni, the altered stool form, bacterial overgrowth and rectal lymphocytes mimic the human condition of IBS.

In addition to irritable bowel syndrome (IBS), another phenomenon linked to IBS is non-ulcer dyspepsia (NUD). This is a condition whereby subjects experience discomfort in the upper abdominal area that cannot be explained by findings on an endoscopy such as an ulcer or irritation of the lining of stomach or intestine. This condition is another of the functional bowel conditions. There is a general recognition that very often there is an overlap between IBS and NUD to a degree that is more than just common occurrence (Talley et al., The association between non-ulcer dyspepsia and other gastrointestinal disorders. SCANS J GASTROENTEROL 1985; 20:896-900). In addition, recent evidence suggests that acute gastroenteritis can precipitate IBS and NUD (Mearin et al., Dyspepsia and irritable bowel syndrome after a Salmonella and gastroenteritis outbreak: One year follow up cohort study. GASTROENTEROL 2005; 129:98-104.). This evidence suggests that the pathophysiology of IBS and NUD may be linked to this initial food poisoning insult. As such, it is likely that the same mechanisms are in play.

The C. jejuni rat model is used herein to test the hypothesis that prophylactic antibiotics may reduce the development of chronic altered bowel form and function after infection. Described herein, it is seen that, in fact, rifaximin prophylaxis reduced the long term effects of C. jejuni on the bowel. This was seen in both stool form and (:)/0 wet weight (Table 2).

Rifaximin is a non-absorbed antibiotic that is approved in the U.S. for the treatment of acute traveler's diarrhea with E. coli (15). In addition to the beneficial effects of rifaximin as an acute treatment, rifaximin has recently gained attention in the prophylaxis of traveler's diarrhea (13). There is one limitation to rifaximin. This limitation is that it is not as effective in the treatment of established acute diarrhea from invasive pathogens such as Shigella or Campylobacter. The presumption is that the lack of absorption of rifaximin limits its access to already invaded organisms. However, a recent study demonstrated that rifaximin was able to prevent illness in humans through prophylaxis of a Shigella infection (16).

Interestingly, though rifaximin appeared effective in preventing the long term altered bowel function, it did not prevent C. jejuni from being detected in stool of rats in the acute phase. The reason(s) why rifaximin did not prevent colonization is not clear. However, without wishing to be bound to any particular theory, the inventors believe that one possible explanation is that rifaximin prevented the invasion of the gut mucosa or minimized the damaging effect on the gut. Thus, bacteria may have been shed quickly and early into the stool. Another explanation stems from studies that suggest rifaximin has its most prominent effect on the small bowel. Perhaps the stool detection of C. jejuni is purely a distal bowel colonization and not small bowel.

The present invention is based on the inventors' finding that rifaximin prophylaxis during acute infection with Campylobacter jejuni, mitigates the long term irregular bowel pattern seen in a post-infectious rat model of IBS, and in effect prevents or reduces the subject's likelihood of developing or having IBS. The inventors further believe that rifaximin prophylaxis will also prevent or reduce a subject's likelihood of developing or having non-ulcer dyspepsia (NUD), based on the evidence discussed above.

The present invention provides for methods of preventing IBS, preventing long term irregular bowel pattern, reducing the likelihood of developing or having IBS, reducing the likelihood of developing or having long term irregular bowel pattern, mitigating IBS that may develop, mitigating long term irregular bowel pattern and reducing the likelihood of developing or having NUD. By mitigating, it is meant that if IBS or long term irregular bowel pattern develops in the subject, the IBS or long term irregular bowel pattern will not be as severe as compared to if the subject did not receive antibiotic treatment for preventing IBS, preventing long term irregular bowel pattern, reducing the likelihood of developing or having IBS, reducing the likelihood of developing or having long term irregular bowel pattern, mitigating IBS that may develop, and/or mitigating long term irregular bowel pattern. For example, the symptoms of IBS (e.g., diarrhea, bloating, constipation) in a treated subject will not be as severe as the symptoms of IBS in an untreated subject.

In one embodiment, the method prevents IBS that results from or is caused by an infection (“post-infectious IBS”), prevents long term irregular bowel pattern that results from or is caused by an infection (“post-infectious long term irregular bowel pattern”), reduces the likelihood of developing or having post-infectious IBS, reduces the likelihood of developing or having post-infectious long term irregular bowel pattern, mitigates post-infectious IBS that may develop, and/or mitigates post-infectious long term irregular bowel pattern. In one embodiment, the infection is an intestinal infection. The intestinal infection may be caused by gastrointestinal pathogens including but not limited to bacteria, viruses, parasites, and amoebas. Examples of bacterial infections include but are not limited to Campylobacter, Escherichia coli (e.g., enterotoxigenic E. coli (ETEC), enterohaemorrhagic E. coli (EHEC), enteropathogenic E. coli (EPEC)), Salmonella, Shigella, and Clostridium difficile. In a particular embodiment, the infection is a C. jejuni infection. Examples of viral infections include but are not limited to rotoviruses and noroviruses. Examples of parasites include but are not limited to trichinella spiralis, giardia and antamoeba.

In one embodiment, the method comprises providing an antibiotic and administering the antibiotic to a subject in need thereof. In another embodiment, the method further comprises identifying a subject who desires the prevention of IBS and/or long term irregular bowel pattern, the reduction in the likelihood of having or developing IBS and/or long term irregular bowel pattern, and/or the mitigation of IBS that may develop and/or long term irregular bowel pattern, and/or the reduction of the likelihood of developing or having NUD, or identifying the subject who needs the prevention of IBS and/or long term irregular bowel pattern, the reduction in the likelihood of having or developing IBS and/or long term irregular bowel pattern, the mitigation of IBS that may develop and/or long term irregular bowel pattern and/or the reduction of the likelihood of developing or having NUD. In one embodiment, the antibiotic is administered to the subject before an intestinal infection (e.g., to a subject without an intestinal infection). In another embodiment, the antibiotic is administered to the subject at the onset of the intestinal infection (e.g., a course of antibiotics may be started at the initial onset of the intestinal infection). In another embodiment, the antibiotic is administered to the subject during the intestinal infection. In another embodiment, the antibiotic is administered to the subject before an intestinal infection and for a period of time that the subject is susceptible to an intestinal infection. In an alternative embodiment, the antibiotic is administered to a subject who has had an intestinal infection.

The subject in need may be any subject who desires the prevention of IBS and/or long term irregular bowel pattern, the reduction of the likelihood of having or developing IBS and/or long term irregular bowel pattern, and/or the mitigation of IBS that may develop and/or long term irregular bowel pattern and/or the reduction of the likelihood of developing or having NUD. In one embodiment, the subject is a subject who has not taken an antibiotic (e.g., rifaximin) to treat an intestinal infection, to prevent an intestinal infection or to reduce the likelihood of an intestinal infection (e.g., acute gastroenteritis, traveler's diarrhea). In another embodiment, the subject may be a subject who has not taken an antibiotic (e.g., rifaximin) to treat a gastric infection, to prevent a gastric infection or to reduce the likelihood of a gastric infection (e.g., acute gastroenteritis).

In another embodiment, the subject does not have small intestinal bacterial overgrowth (SIBO).

In other embodiments, the subject is one who has a genetic predisposition to having IBS. One of ordinary skill in the art will be able to determine subjects who are genetically predisposed to have IBS by performing tests known in the art.

The subject treated with antibiotics in accordance with embodiments of the present invention can depend on the purpose and/or circumstances (e.g., travel, natural disasters, breakdown in sanitation, breakdown in public health, outbreak in a family, and outbreak in a daycare center) as described in more detail below. Thus, in some embodiments, the subject is one who is in one or more of these circumstances. In another embodiment, the subject is one who intends to travel to or is at a location that places the subject at a higher risk of having food poisoning or acute gastroenteritis (e.g., a higher likelihood of ingesting pathogenic gastrointestinal bacteria).

In one particular embodiment, the antibiotic administered is rifaximin. In another particular embodiment, the antibiotics administered are rifaximin and neomycin. In various embodiments, the antibiotic administered may be any antibiotic known in the art. Examples of antibiotics include but are not limited to aminoglycosides (e.g., amikacin, gentamicin, kanamycin, neomycin, netilmicin, streptomycin, tobramycin, paromomycin), ansamycins (e.g., geldanamycin, herbimycin), carbacephems (e.g., loracarbef), carbapenems (e.g., ertapenem, doripenem, imipenem, cilastatin, meropenem), cephalosporins (e.g., first generation: cefadroxil, cefazolin, cefalotin or cefalothin, cefalexin; second generation: cefaclor, cefamandole, cefoxitin, cefprozil, cefuroxime; third generation: cefixime, cefdinir, cefditoren, cefoperazone, cefotaxime, cefpodoxime, ceftazidime, ceftibuten, ceftizoxime, ceftriaxone; fourth generation: cefepime; fifth generation: ceftobiprole), glycopeptides (e.g., teicoplanin, vancomycin), macrolides (e.g., azithromycin, clarithromycin, dirithromycin, erythromycin, roxithromycin, troleandomycin, telithromycin, spectinomycin), monobactams (e.g., aztreonam), penicillins (e.g., amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin), antibiotic polypeptides (e.g., bacitracin, colistin, polymyxin b), quinolones (e.g., ciprofloxacin, enoxacin, gatifloxacin, levofloxacin, lomefloxacin, moxifloxacin, norfloxacin, ofloxacin, trovafloxacin), rifamycins (e.g., rifampicin or rifampin, rifabutin, rifapentine, rifaximin), sulfonamides (e.g., mafenide, prontosil, sulfacetamide, sulfamethizole, sulfanilamide, sulfasalazine, sulfisoxazole, trimethoprim, trimethoprim-sulfamethoxazole (co-trimoxazole, “tmp-sm×”), and tetracyclines (e.g., demeclocycline, doxycycline, minocycline, oxytetracycline, tetracycline) as well as arsphenamine, chloramphenicol, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin/dalfopristin combination, and timidazole.

Particularly effective antibiotics may be non-absorbable antibiotics. Examples of non-absorbable antibiotics include but are not limited to rifaximin, neomycin, Bacitracin, vancomycin, teicoplanin, ramoplanin, and paramomycin.

The regimen for antibiotic administration may depend on the purpose and circumstances (e.g., travel, natural disasters, breakdown in sanitation, breakdown in public health, outbreak in a family, and outbreak in a daycare center). For instance, a subject may be administered a course of antibiotics during the period when there is a risk of exposure to or infection by the bacteria, viruses, or parasites. For example, if an individual goes on a vacation for 10, 20, or 30 days, the antibiotics may be administered before the vacation and/or for the duration of the vacation (e.g., for about 10, 20, or 30 days); during a natural disaster (e.g., hurricane, earthquake) when bacteria, viruses, or parasites enter the water or food supply, a subject may be administered a course of antibiotics until the sanitation conditions return to normal; when one family member in a household is infected by the bacteria, viruses, or parasites, the infected subject and other members of the household can be treated with antibiotics until everyone in the household is cleared of the infection; when a child at a daycare center is infected by bacteria, viruses, or parasites, the child, the caretakers and other children can be treated with antibiotics until everyone in the daycare center is cleared of the infection. These are merely a few examples of circumstances wherein the antibiotics may be administered for preventing IBS, preventing long term irregular bowel pattern, reducing the likelihood of developing or having IBS, reducing the likelihood of developing or having long term irregular bowel pattern, mitigating IBS that may develop, mitigating long term irregular bowel pattern, and reducing the likelihood of developing or having NUD. One of ordinary skill in the art will be able to readily determine additional circumstances wherein administration of antibiotics is appropriate. In other embodiments, the antibiotic may be administered for a course of 3 days, 5 days, 7 days, 10 days, 14 days, three weeks or four weeks. During the course of antibiotics, the antibiotic may be administered once a day, twice a day, three times a day or four times a day. The number of days and frequency per day may depend on the specific antibiotic or antibiotics used and one of ordinary skill in the art may determine an appropriate dosing regimen without undue experimentation.

In various embodiments, 400 mg of rifaximin may be administered three times a day for 10 days; 200 mg of rifaximin may be administered once per day for three days; and 550 mg of rifaximin may be administered once per day for three to ten days.

In one embodiment, a course of antibiotics (e.g., non-absorbable antibiotics, particularly neomycin and/or rifaximin; metronidazole; or vancomycin), may be administered to a subject to prevent or reduce the likelihood of Clostridium difficile infection and thereby prevent IBS, prevent long term irregular bowel pattern, reduce the likelihood of developing or having IBS, reduce the likelihood of developing or having long term irregular bowel pattern, mitigate IBS that may develop, and/or mitigate long term irregular bowel pattern. C. difficile, which naturally resides in the body, can become overgrown as a result from eradication of the normal gut flora by antibiotics, and is a cause of antibiotic-associated diarrhea (AAD). The overgrowth of C. difficile is harmful because the bacteria release toxins that can cause bloating, constipation, and diarrhea with abdominal pain, which may become severe. As such, concurrent treatment with an antibiotic to prevent or reduce the likelihood of Clostridium difficile infection in order to prevent IBS, prevent long term irregular bowel pattern, reduce the likelihood of developing or having IBS, reduce the likelihood of developing or having long term irregular bowel pattern, mitigate IBS that may develop, and/or mitigate long term irregular bowel pattern is contemplated.

In various embodiments, the method comprises, identifying a subject who is being treated with a first antibiotic or will be treated with the first antibiotic; and administering a second antibiotic to prevent or reduce the likelihood of having a Clostridium difficile infection. In various embodiments, the second antibiotic may be a non-absorbable antibiotic, particularly neomycin and/or rifaximin; metronidazole; or vancomycin.

The present invention also provides for methods of inhibiting the production of cytolethal distending toxin (CDT), comprising providing an antibiotic as described above, and administering the antibiotic to a subject. The method may further comprise identifying a subject in need of the inhibition of the production of CDT. The subject may be ones as described above. Inhibiting the production of CDT can be beneficial in preventing IBS, preventing long term irregular bowel pattern, reducing the likelihood of developing or having IBS, reducing the likelihood of developing or having long term irregular bowel pattern, mitigating IBS that may develop, mitigating long term irregular bowel pattern and reducing the likelihood of developing or having NUD. While not wishing to be bound by any particular theory, the inventors believe that CDT found in bacteria, particularly, C. jejuni, is a cause of chronic altered bowel function. CDT, particularly CdtB, is universal among bacteria that causes food poisoning (e.g., Campylobacter (e.g., C. jejuni, C. coli), Escherichia coli (e.g., enterotoxigenic E. coli (ETEC), enterohaemorrhagic E. coli (EHEC), enteropathogenic E. coli (EPEC)), Salmonella, Shigella, and Clostridium difficile), Thus, inhibiting the production of CDT (e.g., by killing the bacteria that produces CDT) can be beneficial for the aforementioned treatments.

The present invention also provides for methods of inhibiting the interaction of CDT with intestinal cells, comprising providing an antibiotic as described above, and administering the antibiotic to a subject. The method may further comprise identifying a subject who is in need of the inhibition of the interaction of CDT with intestinal cells. The subject may be ones as described above. Inhibiting the interaction of CDT with intestinal cells can be beneficial in preventing IBS, preventing long term irregular bowel pattern, reducing the likelihood of developing or having IBS, reducing the likelihood of developing or having long term irregular bowel pattern, mitigating IBS that may develop, mitigating long term irregular bowel pattern, and reducing the likelihood of developing or having NUD.

In various embodiments, the subject treated by methods of the present invention is a mammalian subject; particularly, a human subject.

In various embodiments, the present invention provides pharmaceutical compositions including a pharmaceutically acceptable excipient along with a therapeutically effective amount of the antibiotic. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous.

In various embodiments, the pharmaceutical compositions according to the invention may be formulated for delivery via any route of administration. “Route of administration” may refer to any administration pathway known in the art, including but not limited to aerosol, oral, parenteral, or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Via the parenteral route, the compositions may be in the form of solutions or suspensions for infusion or for injection.

The pharmaceutical compositions according to the invention can also contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Typical dosages of an effective antibiotic can be in the ranges recommended by the manufacturer where known therapeutic compounds are used, and also as indicated to the skilled artisan by the responses in animal models. Such dosages typically can be reduced by up to about one order of magnitude in concentration or amount without losing the relevant biological activity. Thus, the actual dosage can depend upon the judgment of the physician, the condition of the patient, and the effectiveness of the therapeutic method based, for example, on the responses observed in the appropriate animal models, as previously described.

The present invention is also directed to a kit for preventing IBS, preventing long term irregular bowel pattern, reducing the likelihood of developing or having IBS, reducing the likelihood of developing or having long term irregular bowel pattern, mitigating IBS that may develop, and/or mitigating long term irregular bowel pattern. The kit is an assemblage of materials or components, including at least an antibiotic, as described above.

The exact nature of the components configured in the inventive kit depends on its intended purpose. For example, various embodiments are configured for the purposes of preventing IBS, preventing long term irregular bowel pattern, reducing the likelihood of developing or having IBS, reducing the likelihood of developing or having long term irregular bowel pattern, mitigating IBS that may develop, mitigating long term irregular bowel pattern and/or reducing the likelihood of developing or having NUD. In one embodiment, the kit is configured particularly for mammalian subjects. In another embodiment, the kit is configured particularly for human subjects. In further embodiments, the kit is configured for veterinary applications, for subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to prevent IBS, prevent long term irregular bowel pattern, reduce the likelihood of developing or having IBS, reduce the likelihood of developing or having long term irregular bowel pattern, mitigate IBS, mitigate long term irregular bowel pattern. Optionally, the kit also contains other useful components, such as, diluents, buffers, pharmaceutically acceptable carriers, syringes, catheters, applicators, pipetting or measuring tools or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in the treatment of IBS or infections. As used herein, the term “package” refers to a suitable solid matrix or material such as glass, plastic, paper, foil, and the like, capable of holding the individual kit components. Thus, for example, a package can be a plastic bottle used to contain suitable quantities of an antibiotic. The packaging material generally has an external label which indicates the contents and/or purpose of the kit and/or its components.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Animal Preparation and Gavage

Male Sprague-Dawley rats (n=108) (200g) were quarantined for 10 days. Subsequently, fresh stool was collected by stroking the anus. Stool was cultured for C. jejuni on Campylobacter agar (BD Diagnostics, Franklin Lakes, N.J.). This was conducted to confirm the absence of infection or colonization of animals with C. jejuni prior to starting the study.

Once it was confirmed that the rats did not harbor C. jejuni, the rats were divided into two groups. In the first group (n=54), the rats were gavaged with 1 mL of a 5% solution of bicarbonate. This was given to acutely reduce gastric acidity. Subsequently, the rats were gavaged with a 1 mL suspension of 10⁸ cfu/mL C. jejuni 81-176 in Campylobacter broth (C+/R− group). In the second group (n=54), the rats were gavaged with a 1 mL solution of rifaximin (200 mg) (C+/R+ group). The following day, the C+/R+ group received 3 sequential gavages. The first was another 1 mL dose of rifaximin. One hour later, they were gavaged with a 1 mL solution of 5% bicarbonate followed by a third gavage consisting of a 1 mL suspension of 10⁸ cfu/mL C. jejuni 81-176 in Campylobacter broth. The following day, the C+/R+ group received a third and final gavage of 200 mg of rifaximin.

### Example 2

Tracking the Acute Colonization with C. jejuni

After completion of the gavage, fresh stool was collected daily from all rats (both groups) and cultured for the presence of C. jejuni on Campylobacter agar. The number of days to first detectable C. jejuni in stool was recorded. Once colonization was noted, daily stool was cultured until 2 consecutive days with no detectable C. jejuni was observed. This was recorded as the time to C. jejuni clearance.

### Example 3

### Determination of a Post-Infectious Phenotype

After C. jejuni clearance was determined, the rats were housed for an additional 3 months (90 days). At 90 days, fresh stool was collected for 3 consecutive days. The stool was graded for consistency. To do this, a modified Bristol-like stool score was used. In this score, stool was graded as normal (1), soft and poorly formed (2), or watery (3). The stool was then weighed and air dried for 5 days followed by oven drying at 160° C. for 1 hour. The stool was reweighed and a % stool wet weight was determined. After completion of the study, the rats were euthanized by CO₂ asphyxiation, weighed, and a laparotomy was performed. During the laparotomy, predefined segments of duodenum, jejunum, ileum, left colon, and rectum were resected, as previously described (12). Subsequently, the contents of each segment were cultured for the presence of C. jejuni.

### Example 4

### Data Analysis

In the post-infectious phase, the time to initial colonization, time for clearance and total duration of shedding were determined and compared between the C+/R− and C+/R+ groups. This was compared by Wilcoxon rank-sum test.

At the end of 3 months, the stool form for 3 days was averaged and then the two groups were compared. In another analysis, the number of days with normal stool in 3 days was determined and compared between groups. Finally, the variance in stool form over 3 days was also determined and compared between groups. The comparison was done by Wilcoxon rank-sum test.

### Example 5

### Campylobacter Colonization Phase

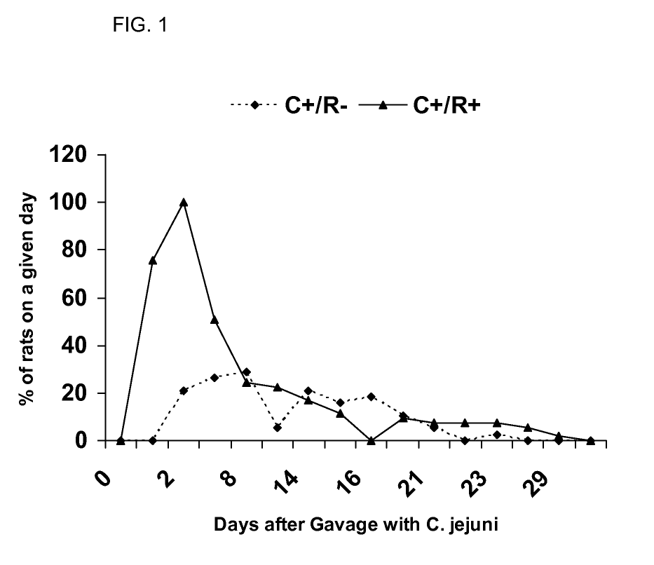

Out of the 108 rats initially in the study, none were found to have C. jejuni in stool prior to gavage with C. jejuni 81-176. A total of 9 rats died in the 3 days after gavage (8 due to gavage trauma and 1 due to a bleeding liver lesion that appeared to be an unrecognized tumor). All were in the C+/R− group. The remaining rats survived all stages of gavage and there was no clinical evidence of trauma from gavage. In FIG. 1, the colonization dynamics are depicted by day after gavage. Of the 45 C+/R− rats, 38 (84%) were colonized with C. jejuni in their stool on at least 1 day following gavage. This was different from the C+/R+ group. In the C+/R+ group of 54 rats, 53 (98%) of the rats had at least one day of detectable C. jejuni (P=0.016). Between groups, there was also a difference in the time to first detectable colonization such that the C+/R+ group had detectable colonization with C. jejuni sooner than the C+/R− group (P<0.0001) (Table 1). However, mean day to clearance of C. jejuni in the C+/R+ arm was sooner than the rats in C+/R− (P<0.01). The longest colonization was 27 days and was seen in the C+/R− group. The longest colonization in the C+/R+ group was 21 days.

TABLE 1
Colonization times in rats with and without rifaximin prophylaxis.

| | C+/R− | C+/R+ | P-value |
|---|---|---|---|
| Time to first detectable C. jejuni | 6.7 ± 4.5 | 1.3 ± 0.4 | <0.0001 |
| Time to clearance of C. jejuni | 12.6 ± 5.9 | 10.3 ± 7.1 | <0.01 |

### Example 6

### Post-infectious Phase of Study

Three months following clearance of C. jejuni, stool form and % wet weight were notably different between groups (Table 2). Although the 3 day average wet weight was not significantly different between groups, within those 3 days the stool was variable in form and wet weight. This is evidenced by the significant difference in the variability of the stool % wet weight over the 3 days. The larger the variability, the more variable the stool was from day to day in terms of % wet weight. The C+/R− group had a wider range of stool moisture over 3 days as expressed by 3 day wet weight variance (P<0.01).

Most revealing was the stool consistency. As seen in table 2, the average stool consistency was significantly less formed in the C+/R− group as compared to the group that received rifaximin (P<0.01). Once again, the variability in stool form produced an even greater difference.

TABLE 2
Post-infectious stool parameters in rats with (C+/R+) and
without (C+/R−) rifaximin prophylaxis.

| | | Campy | Rifaximin | |
|---|---|---|---|---|
| Stool Parameter | Control | (C+/R−) | (C+/R+) | P-value |
| Average % of stool | 63.7 +/− 3.2 | 60.1 +/− 6.8 | 61.1 +/− 3.8 | NS |
| dry weight | | | | |
| Daily variability of | 4.9 +/− 3.8 | 8.4 +/− 6.4 | 4.1 +/− 2.3 | <0.01 |
| Dry Weight | | | | |
| Average Stool Consistency | 1.0 +/− 0.0 | 1.5 +/− 0.4 | 1.1 +/− 0.3 | <0.00001 |
| (Based on Bristol Stool | | | | |
| Scale) | | | | |
| Daily Variability of | 0.0 | 0.51 +/− 0.38 | .24 +/− 0.5 | <0.01 |
| Stool Consistency | | | | |

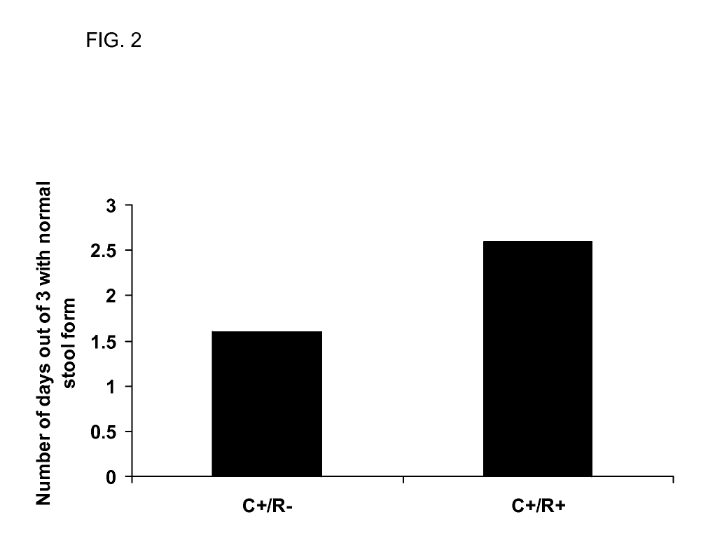

FIG. 2 is another depiction of the stool form. Out of 3 days, the rats that received rifaximin (C+/R+) were closer to having perfectly normal bowel function (i.e., closer to 3 normal bowel days) than the group that had not received rifaximin prophylaxis (C+/R−).

### Example 7

### Post-Mortem Analysis

After euthanasia, live C. jejuni could not be found in any animal 3 months after clearance in stool. This included culture of the duodenum, jejunum, ileum, left colon and stool.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

### REFERENCES

1. McKendrick M W, Read N W. Irritable bowel syndrome—post salmonella infection. J Infect 1994; 29:1-3.
2. Neal K R, Hebden J, Spiller R. Prevalence of gastrointestinal symptoms six months after bacterial gastroenteritis, and risk factors for development of the irritable bowel syndrome: postal survey of patients. BMJ 1997; 314: 779-782.
3. Gwee K A, Leong Y L, Graham C, et al. The role of psychological and biological factors in postinfective gut dysfunction. Gut 1999; 44:400-6
4. Parry S D. Stansfield R. Jelley D. Gregory W. Phillips E. Barton J R. Welfare M R. Is irritable bowel syndrome more common in patients presenting with bacterial gastroenteritis? A community-based, case-control study. Am J Gastroenterol. 2003 February; 98(2):238-41.
5. Barber R, Blakey A. Prevalence of gastrointestinal symptoms after bacterial gastroenteritis: study did not include control group. BMJ 1997; 314:1903.
6. Neal K R, Barker L, Spiller R C. Prognosis in post-infectious irritable bowel syndrome: a six year follow up study. Gut 2002; 51:410-13.
7. Okhuysen P C, Jiang Z D, Carlin L, et al. Post-diarrhea chronic intestinal symptoms and irritable bowel syndrome in North American travelers to Mexico. Am J Gastroenterol 2004; 99:1774-8.
8. Mearin F, Perez-Oliveras M, Perello A, et al. Dyspepsia and irritable bowel syndrome after a Salmonella outbreak: one-year follow-up cohort study. Gastroenterol 2055; 129:98-104.
9. Halvorson H A, Schlett C D, Riddle M S. Postinfectious irritable bowel syndrome—a meta-analysis. Am J Gastroenterol 2006; 101:1894-9.
10. Neal K R, Barker L, Spiller R C. Prognosis in post-infectious irritable bowel syndrome: a six year follow up study. Gut 2002; 51:410-13.
11. Spiller R C, Jenkins D, Thornley J P, et al. Increased rectal mucosal enteroendocrine cells, T lymphocytes, and increased gut permeability following acute Campylobacter enteritis and in post-dysenteric irritable bowel syndrome. Gut. 2000; 47:804-11.
12. Pimentel M, Chatterjee S, Chang C, Low K, Song Y, Liu C, Lezcano S, Conklin J, Finegold S. A new rat model links two contemporary theories in irritable bowel syndrome. Dig Dis Sci 2008; 53:982-9.
13. Cabada M M, White A C Jr. Traveler's diarrhea: an update on susceptibility, prevention and treatment. Curr Gastroenterol Rep 2008; 10:473-9.
14. Bercik P, Wang L, Verdú E F, et al. Visceral hyperalgesia and intestinal dysmotility in a mouse model of postinfective gut dysfunction. Gastroenterology 2004; 127:179-87.
15. Baker D E: Rifaximin: a nonabsorbed oral antibiotic. Rev Gastroenterol Disord 2005; 5:19-30.
16. Taylor D N, McKenzie R, Durbin A, et al. Rifaximin, a non-absorbed oral antibiotic, prevents shigellosis after experimental challenge. Clin Infect Dis 2006; 42:1283-8.

What is claimed is:

1. A method, comprising:
identifying a subject selected from the group consisting of: a subject who desires a reduction of the likelihood of developing or having post infectious irritable bowel syndrome (PI-IBS), a subject who desires a reduction of the likelihood of developing or having long term irregular bowel pattern, a subject who desires a mitigation of PI-IBS that may develop, a subject who desires a mitigation of long term irregular bowel pattern, a subject who desires a reduction of the likelihood of developing or having non-ulcer dyspepsia (NUD), a subject in need of reducing the likelihood of developing or having post infectious irritable bowel syndrome (PI-IBS), a subject in need of reducing the likelihood of developing or having long term irregular bowel pattern, a subject in need of mitigating PI-IBS that may develop, a subject in need of mitigating long term irregular bowel pattern, a subject in need of reducing the likelihood of developing or having NUD, and combinations thereof;
providing an antibiotic; and
administering the antibiotic to the subject to reduce the likelihood of developing or having PI-IBS, to reduce the likelihood of developing or having long term irregular bowel pattern, to mitigate PI-IBS that may develop, to mitigate long term irregular bowel pattern and/or to reduce the likelihood of developing or having NUD in the subject.

2. The method of claim 1, wherein the antibiotic is a non-absorbable antibiotic.

3. The method of claim 2, wherein the non-absorbable antibiotic is rifaximin.

4. The method of claim 1, wherein the subject does not have small intestinal bacterial overgrowth (SIBO).

5. The method of claim 1, wherein the subject has not taken an antibiotic to treat an intestinal infection, to prevent an intestinal infection, to reduce the likelihood of having an intestinal infection, to treat a gastric infection, to treat a gastric infection, to prevent a gastric infection or to reduce the likelihood of having a gastric infection.

6. The method of claim 1, wherein the subject is exposed to a higher risk of having food poisoning or gastroenteritis.

7. The method of claim 6, wherein the food poisoning or gastroenteritis is caused by Campylobacter.

8. The method of claim 7, wherein the Campylobacter is Campylobacter jejuni.

9. The method of claim 6, wherein the food poisoning or gastroenteritis is caused by Escherichia coli, Salmonella or Shigella.

10. A method, comprising:
identifying a subject in need of inhibiting the production of cytolethal distending toxin (CDT) and/or inhibiting the interaction of CDT with an intestinal cell;
providing an antibiotic; and
administering the antibiotic to the subject to inhibit the production of CDT and/or inhibit the interaction of CDT with the intestinal cell.

11. The method of claim 10, wherein inhibiting the production of CDT and/or inhibiting the interaction of CDT with the intestinal cell reduce the likelihood of developing or having post infectious irritable bowel syndrome (PI-IBS), reduce the likelihood of developing or having long term irregular bowel pattern, mitigate PI-IBS that may develop, mitigate long term irregular bowel pattern, and/or reduce the likelihood of developing or having non-ulcer dyspepsia (NUD).

12. The method of claim 10, wherein the antibiotic is a non-absorbable antibiotic.

13. The method of claim 12, wherein the non-absorbable antibiotic is rifaximin.

14. The method of claim 10, wherein the subject does not have small intestinal bacterial overgrowth (SIBO).

15. The method of claim 10, wherein the subject has not taken an antibiotic to treat an intestinal infection, to reduce the likelihood of having an intestinal infection, to treat a gastric infection, and/or to reduce the likelihood of having a gastric infection.

16. The method of claim 10, wherein the subject is exposed to a higher risk of having food poisoning or gastroenteritis.

17. The method of claim 16, wherein the food poisoning or gastroenteritis is caused by Campylobacter.

18. The method of claim 17, wherein the Campylobacter is Campylobacter jejuni.

19. The method of claim 16, wherein the food poisoning or gastroenteritis is caused by Escherichia coli, Salmonella or Shigella.

20. A method, comprising:
identifying a subject who is being treated with a first antibiotic or will be treated with the first antibiotic; and
administering a second antibiotic selected from the group consisting of rifaximin, neomycin, metronidazole, vancomycin and combinations thereof to reduce the subject's likelihood of having a Clostridium difficile infection.

21. The method of claim 20, wherein reducing the subject's likelihood of having a Clostridium difficile infection reduces the subject's likelihood of developing or having irritable bowel syndrome (IBS), reduces the subject's likelihood of developing or having long term irregular bowel pattern, mitigates IBS that may develop in the subject, mitigates long term irregular bowel pattern for the subject, and/or reduces the likelihood of developing or having non-ulcer dyspepsia (NUD).
